(19) United States
(12) Patent Application Publication
(10) Pub. No.: US 2022/0004774 A1
(43) Pub. Date: Jan. 6, 2022

(54) INFORMATION PROCESSING DEVICE, INFORMATION PROCESSING METHOD, AND INFORMATION PROCESSING SYSTEM

(71) Applicant: TOYOTA JIDOSHA KABUSHIKI KAISHA, Toyota-shi (JP)

(72) Inventors: Shuhei YAMAMOTO, Aichi-gun (JP); Yurika TANAKA, Yokosuka-shi (JP); Satoshi KOMAMINE, Nagoya-shi (JP); Hideo HASEGAWA, Nagoya-shi (JP); Tomoya MATSUBARA, Seto-shi (JP); Ibuki SHIMADA, Miyoshi-shi (JP); Keisuke SHOJI, Nagoya-shi (JP)

(73) Assignee: TOYOTA JIDOSHA KABUSHIKI KAISHA, Toyota-shi (JP)

(21) Appl. No.: 17/360,053

(22) Filed: Jun. 28, 2021

(30) Foreign Application Priority Data
Jul. 1, 2020 (JP) 2020-114026

(51) Int. Cl. G06K 9/00; G06T 7/70; H04N 7/18; G01L 5/00

(52) U.S. Cl. CPC G06K 9/00771; G06K 9/00369; G06K 9/00785; H04W 4/12; H04N 7/18; G01L 5/00; G06T 2207/30236; G06T 7/70

(57) ABSTRACT

An information processing device includes a controller. The controller is configured to detect an obstruction present on a road, is configured to detect a user present within a predetermined distance from the obstruction that is detected, and is configured to transmit, to a terminal of the user that is detected, a request to relocate the obstruction.

### CROSS-REFERENCE TO RELATED APPLICATION

This application claims priority to Japanese Patent Application No. 2020-114026 filed on Jul. 1, 2020, incorporated herein by reference in its entirety.

### BACKGROUND

### 1. Technical Field

The disclosure relates to an information processing device, and information processing method, and an information processing system.

### 2. Description of Related Art

There is known a guidance system for the visually impaired, in which ultrasonic waves are emitted from a guidance sensor worn by the visually impaired, reflected waves are received, obstructions being approached are detected thereby, and annunciation of information necessary for walking is performed by audio or signals (e.g., see Japanese Unexamined Patent Application Publication No. 08-332198 (JP 08-332198 A)).

### SUMMARY

According to the above technology, a pedestrian can circumvent an obstruction, but the obstruction remains in that place, and accordingly other following pedestrians who pass that location need to circumvent that obstruction as well. The present disclosure provides promotion of removal of the obstruction.

A first aspect of the present disclosure is an information processing device including a controller. The controller is configured to detect an obstruction present on a road, is configured to detect a user present within a predetermined distance from the obstruction that is detected, and is configured to transmit, to a terminal of the user that is detected, a request to relocate the obstruction.

In the first aspect, the controller may be configured to acquire an image taken by a camera that takes an image of the road, and may be configured to detect the obstruction based on the image.

In the first aspect, the controller may be configured to acquire a detection value from a sensor configured to detect pressure applied to the road, and may be configured to detect the obstruction based on the detection value from the sensor.

In the first aspect, the controller may be configured to detect the user present within the predetermined distance from the obstruction, by detecting the terminal of the user present within the predetermined distance from the obstruction.

In the first aspect, the controller may be configured to select the terminal of the user that is configured to receive transmission of the request to relocate the obstruction, in accordance with an attribute of the user.

In the first aspect, the controller may be configured to select the terminal of the user that is configured to receive transmission of the request to relocate the obstruction, in accordance with an attribute of the obstruction and an attribute of the user.

In the first aspect, the controller may be configured to transmit information about a reward to the terminal of the user when the obstruction is relocated.

In the first aspect, the controller may be configured to change the predetermined distance in accordance with an amount of traffic of a pedestrian or a vehicle on the road where the obstruction is present.

In the first aspect, the controller may be configured to set the predetermined distance longer as the traffic is heavier.

In the first aspect, the controller may be configured to, when the obstruction is relocated and the controller transmits information about the reward to the terminal of the user, set the reward higher as the traffic is heavier.

In the first aspect, the controller may be configured to change the predetermined distance in accordance with whether a position where the obstruction is present is a predetermined location.

In the first aspect, the predetermined location may be a location at which tactile tiles are installed.

In the first aspect, the controller may be configured to set a first distance as the predetermined distance when the position at which the obstruction is present is the predetermined location. The controller may be configured to set a second distance as the predetermined distance when the position at which the obstruction is present is not the predetermined location. The first distance may be longer than the second distance.

In the first aspect, the controller may be configured to set a first value as the reward when the obstruction is relocated, the controller transmits information about the reward to the terminal of the user, and the position at which the obstruction is present is the predetermined location. The controller may be configured to set a second value as the reward when the obstruction is relocated, the controller transmits information about the reward to the terminal of the user, and the position at which the obstruction is present is not the predetermined location. The first value may be higher than the second value

A second aspect of the present disclosure is an information processing method executed by a computer. The information processing method includes detecting an obstruction present on a road, detecting a user present within a predetermined distance from the obstruction that is detected, and transmitting, to a terminal of the user that is detected, a request to relocate the obstruction.

In the second aspect, the information processing method may further include selecting, by the computer, the terminal of the user that is configured to receive transmission of the request to relocate the obstruction, in accordance with an attribute of the user.

In the second aspect, the information processing method may further include transmitting, by the computer, information about a reward to the terminal of the user when the obstruction is relocated.

A third aspect of the present disclosure is an information processing system. The system includes a sensor configured to output in accordance with an obstruction present on a road, and a server including a controller. The controller is configured to detect the obstruction, based on output of the sensor, is configured to detect a user present within a predetermined distance from the obstruction, and is configured to transmit, to a terminal of the user, a request to relocate the obstruction.

In the third aspect, the controller may be configured to select the terminal of the user that is configured to receive transmission of the request to relocate the obstruction, in accordance with an attribute of the user.

In the third aspect, the controller may be configured to transmit information about a reward to the terminal of the user when the obstruction is relocated.

According to the first aspect, the second aspect, and the third aspect of the present disclosure, removal of an obstruction can be promoted.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram illustrating a schematic configuration of a system according to an embodiment;

FIG. 2 is a diagram for illustrating image-taking performed in the embodiment;

FIG. 3 is a block diagram schematically illustrating an example of configurations of each of a camera, a user terminal, and a server, configuring the system according to the embodiment;

FIG. 4 is a diagram exemplifying a functional configuration of the server;

FIG. 5 is a diagram exemplifying a table configuration of an image database;

FIG. 6 is a diagram exemplifying a table configuration of a user terminal database;

FIG. 7 is a diagram exemplifying a functional configuration of a user terminal;

FIG. 8 is a flowchart of processing of a server according to a first embodiment transmitting a relocation request to a user terminal;

FIG. 9 is a flowchart of processing when the user terminal according to the embodiment receives a relocation request from the server;

FIG. 10 is a diagram exemplifying a table configuration of an image database according to a second embodiment;

FIG. 11 is a diagram exemplifying a table configuration of a user terminal database according to the second embodiment;

FIG. 12 is a flowchart of processing of a server according to the second embodiment transmitting a relocation request to a user terminal;

FIG. 13 is a flowchart of processing of a server according to a third embodiment imparting a reward to a user;

FIG. 14 is a diagram exemplifying a table configuration of an image database according to a fourth embodiment;

FIG. 15 is a flowchart of processing of a server according to the fourth embodiment transmitting a relocation request to a user terminal;

FIG. 16 is a flowchart of processing of the server according to the fourth embodiment imparting a reward to a user; and

FIG. 17 is a block diagram schematically illustrating an example of configurations of each of a pressure detecting device, a user terminal, and a server, configuring a system when determining presence of an obstruction, based on pressure applied to a road.

### DETAILED DESCRIPTION OF EMBODIMENTS

An information processing device that is one aspect of the present disclosure is provided with a control unit. The control unit executes detecting of an obstruction present on a road, detecting a user present within a predetermined distance from the detected obstruction, and transmitting a request to relocate the obstruction to a terminal of the user that is detected. Detection of the obstruction is performed based on detection values of a sensor, for example. Examples of the sensor include an image sensor provided to a camera, and a pressure sensor installed in a road. For example, the obstruction can be detected by analyzing images taken by a camera. Also, in locations where an obstruction is present, for example, the obstruction can be detected, since pressure detected by the pressure sensor installed in the road is greater. Note that a plurality of sensors may be combined for detection of an obstacle.

Also, the control unit detects users present within a predetermined distance from the detected obstruction. The detected user may be selected to be a user to relocate the obstruction. The predetermined distance is a distance that serves as a threshold value regarding whether the user is to be made to relocate the obstruction, for example. That is to say, it is highly possible that a user close to the obstruction relocates the obstruction, but the farther from the obstruction the user is, the lower the possibility that the obstruction is relocated. Accordingly, a distance between the obstruction and the user such that the user can be expected to remove the obstruction is set as the predetermined distance, for example. Thus, it is highly possible that a user present within the predetermined distance from the obstruction relocates the obstruction. The distance between the obstruction and the user can be determined based on the position of the obstruction and the position of the user, for example. The position of the obstruction can be obtained based on the position of the above sensor, for example. Also, the position of the user can be obtained based on the position of the above sensor and the output of the sensor, for example. The position of the user can also be obtained based on signals from the terminal that the user possesses.

The control unit transmits a request to relocate the obstruction to the terminal of the user that is detected. This request may contain information by which the obstruction can be identified. By the user recognizing the request received at the terminal, the user can remove the obstruction. Note that the user may be given a reward.

Embodiments of the present disclosure will be described below with reference to the drawings. Note that the configurations of the embodiments below are exemplary, and that the present disclosure is not limited to the configurations of the embodiments. Also, the following embodiments may be combined in any way insofar as there is no contradiction.

### First Embodiment

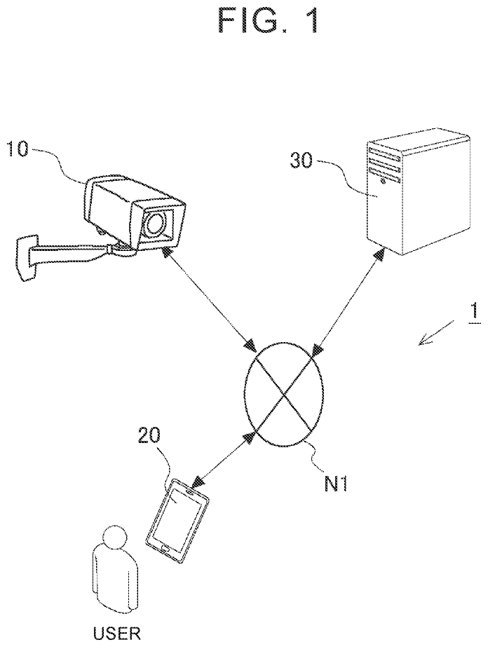

FIG. 1 is a diagram illustrating a schematic configuration of a system 1 according to a first embodiment. The system 1 is a system in which an obstruction on a road is detected by a server 30 analyzing images taken by a camera 10, and a request to relocate the obstruction is transmitted to a user terminal 20 that a user within a predetermined distance from the obstruction possesses.

In the example in FIG. 1, the system 1 includes the camera 10, the user terminal 20, and the server 30. The camera 10, the user terminal 20, and the server 30 are mutually connected by a network N1. The camera 10 is, for example, a surveillance camera, a live camera, or the like. The user terminal 20 is a terminal that a user uses.

The network N1 is a global-scale public communication network such as the Internet or the like, for example, and wide area networks (WAN) or other communication networks may be employed. The network N1 may also include a telephone communication network such as a cellular phone communication network, a wireless communication network such as Wi-Fi (registered trademark), or the like. Although one camera 10 and one user terminal 20 are exemplarily illustrated in FIG. 1, a plurality thereof may exist.

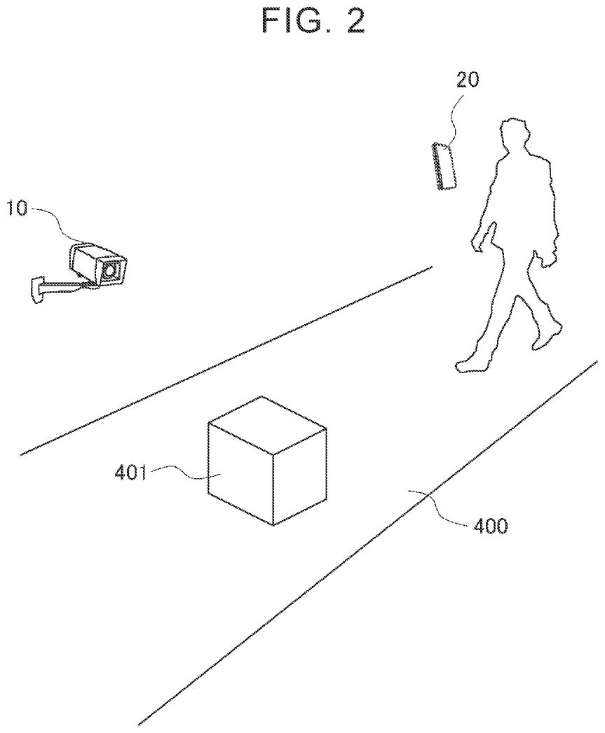

FIG. 2 is a diagram for illustrating taking images, which is performed in the present embodiment. In the present embodiment, the camera 10 takes images of a road 400. Note that the object of taking images by the camera 10 is not limited to the road 400, and may be any location that the user can pass through. Images taken by the camera 10 are transmitted to the server 30. There may be an obstruction 401 that would impede passage of pedestrians in an image taken by the camera 10. The server 30 determines whether the obstruction 401 is in the image, by performing image analysis, for example. Upon detecting the obstruction 401, the server 30 transmits a request to relocate the obstruction 401 to the user terminal 20 of a user nearby. The user terminal 20 that has received this request displays on a display or emits a sound that there has been a request to relocate the obstruction 401, thereby preforming annunciation to the user that there has been a request. The user responds to this request, and thus the user relocates the obstruction 401. Thus, the visually impaired can be suppressed from coming into contact with the obstruction, for example.

Note that an arrangement may be made in which the server 30 transmits a request for relocation of the obstruction 401 only when there is an obstruction 401 on tactile tiles. That is to say, an arrangement may be made in which the server 30 transmits a request for relocation of the obstruction 401 only when there is the obstruction 401 with which there is a possibility that the visually impaired might come into contact with. Whether there is an obstruction 401 on tactile tiles can be determined by analyzing images taken by the camera 10, for example.

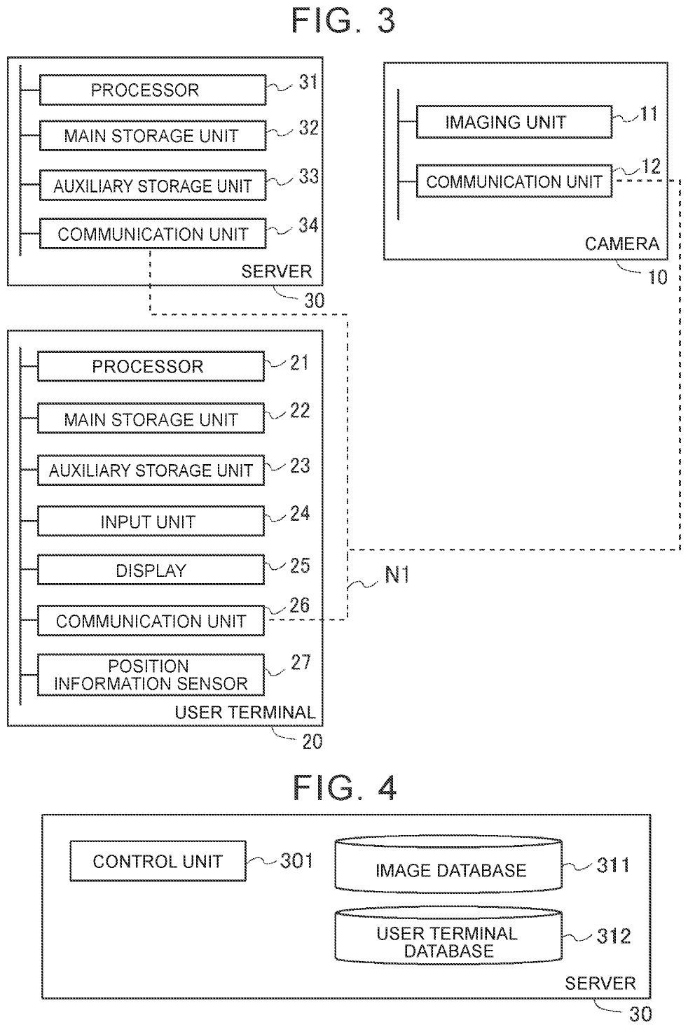

The hardware configuration and the functional configuration of the camera 10, the user terminal 20, and the server 30 will be described based on FIG. 3. FIG. 3 is a block diagram schematically illustrating an example of the configuration of each of the camera 10, the user terminal 20, and the server 30, configuring the system 1 according to the present embodiment.

The server 30 has the configuration of a common computer. The server 30 has a processor 31, a main storage unit 32, an auxiliary storage unit 33, and a communication unit 34. These are mutually connected by a bus. The processor 31 is an example of a control unit.

The processor 31 is a central processing unit (CPU), a digital signal processor (DSP), or the like. The processor 31 controls the server 30 to perform various types of information processing computations. The processor 31 is an example of a control unit. The main storage unit 32 is random access memory (RAM), read-only memory (ROM), or the like. The auxiliary storage unit 33 is erasable programmable ROM (EPROM), a hard disk drive (HDD), removable media, or the like. An operating system (OS), various types of programs, various types of tables, and so forth, are stored in the auxiliary storage unit 33. The processor 31 loads programs stored in the auxiliary storage unit 33 to a work region of the main storage unit 32 and executes the programs, and the components and so forth are controlled by this execution of programs. Thus, the server 30 realizes functions matching predetermined objects. The main storage unit 32 and the auxiliary storage unit 33 are computer-readable storage media. Note that the server 30 may be a single computer, or may be a collaboration of a plurality of computers. Also, information stored in the auxiliary storage unit 33 may be stored in the main storage unit 32. Also, information stored in the main storage unit 32 may be stored in the auxiliary storage unit 33.

The communication unit 34 is means that performs communication with the camera 10 and the user terminal 20 via the network N1. The communication unit 34 is, for example, a local area network (LAN) interface board, or a wireless communication circuit for wireless communication. The LAN interface board and the wireless communication circuit are connected to the network N1.

Next, the camera 10 is a device that is installed indoors or outdoors and takes images in the vicinity of the camera 10. The camera 10 is provided with an imaging unit 11 and a communication unit 12. The imaging unit 11 uses an imaging device such as a charge-coupled device (CCD) image sensor or a complementary metal-oxide semiconductor (CMOS) image sensor, or the like, for example, to take images. Images obtained by image-taking may be either still images or moving images.

The communication unit 12 is communication means for connecting the camera 10 to the network N1. The communication unit 12 is a circuit for performing communication with other devices (e.g., the server 30 or the like) via the network N1, using wireless communication such as a mobile communication service (e.g., a telephone communication network such as 5th generation (5G), 4th generation (4G), 3rd generation (3G), Long-Term Evolution (LTE), and so forth), Wi-Fi (registered trademark), Bluetooth (registered trademark), or the like, for example. Images taken by the camera 10 are transmitted to the server 30 via the communication unit 12.

Next, the user terminal 20 will be described. The user terminal 20 is, for example, a small-sized computer, such as a smartphone, a cellular phone, a tablet terminal, a personal information terminal, a wearable computer (smart watch or the like), a personal computer (PC), or the like. The user terminal 20 has a processor 21, a main storage unit 22, an auxiliary storage unit 23, an input unit 24, a display 25, a communication unit 26, and a position information sensor 27. These components are mutually connected by a bus. Description of the processor 21, the main storage unit 22, and the auxiliary storage unit 23 will be omitted, since these are the same as the processor 31, the main storage unit 32, and the auxiliary storage unit 33, of the server 30.

The input unit 24 is means to accept input operations performed by the user, examples of which include a touchpad, a mouse, a keyboard, pushbuttons, and so forth. The display 25 is means to present information to the user, examples of which include a liquid crystal display (LCD), an electroluminescence (EL) panel, and so forth. The input unit 24 and the display 25 may be integrally formed to configure a single touchscreen panel. The communication unit 26 is communication means for connecting the user terminal 20 to the network N1. The communication unit 26 is a circuit for performing communication with other devices (e.g., the server 30 or the like) via the network N1, using a wireless communication network such as a mobile communication service (e.g., a telephone communication network such as 5G, 4G, 3G, LTE, and so forth), Wi-Fi (registered trademark), Bluetooth (registered trademark), or the like, for example.

The position information sensor 27 acquires position information of the user terminal 20 (e.g., latitude and longitude) at predetermined cycles. The position information sensor 27 is, for example, a Global Positioning System (GPS) receiver, a wireless communication unit, or the like. Information acquired by the position information sensor 27 is recorded in the auxiliary storage unit 23 or the like, for example, and is transmitted to the server 30.

Next, the functions of the server 30 will be described. FIG. 4 is a diagram exemplifying the functional configuration of the server 30. The server 30 is provided with a control unit 301, an image database 311, and a user terminal database 312, as functional components. The processor 31 of the server 30 executes the processing of the control unit 301 by a computer program in the main storage unit 32. The image database 311 and the user terminal database 312 are constructed by a program of a database management system (DBMS) executed by the processor 31 managing data stored in the auxiliary storage unit 33. The image database 311 and the user terminal database 312 are, for example, relational databases. Note that part of the functional components of the server 30, or part of the processing thereof, may be executed by another computer connected to the network N1.

The control unit 301 receives images from the camera 10, and stores the images in the image database 311. Also, the control unit 301 performs image analysis of the images stored in the image database 311, and extracts images in which there is an obstruction on the road 400. Related arts can be used for this extraction. For example, when comparing an image taken last time and an image taken this time and there are portions on the road 400 that are different, an obstruction 401 may be determined to exist there. Also, the type of the object in the image may be identified by performing image analysis, and determination may be made regarding whether the object is an obstruction 401.

Also, the control unit 301 acquires position information of the obstruction 401. Note that the position of the camera 10 may be handled as the position of the obstruction 401. The position of the camera 10 may be input to the image database 311 in advance that is associated with an identification symbol of the camera 10 (camera ID). That is to say, the position where the image was taken may be acquired by registering the position of the camera 10 in the server 30 in advance, and identifying the camera 10 transmitting the image. Alternatively, position information of the camera 10 may be included in images transmitted from the camera 10 to the server 30, for example. Alternatively again, the control unit 301 may identify the position of the obstruction 401 based on the position of the camera 10, the angle of the camera 10, and the position of the obstruction 401 in the image.

The control unit 301 also identifies user terminals 20 present within a predetermined distance from the obstruction 401. The control unit 301 stores position information transmitted from the user terminals 20 in the user terminal database 312. Position information is emitted from the user terminals 20 every predetermined amount of time, and accordingly the control unit 301 updates the user terminal database 312 each time. The position information is emitted from the user terminals 20 while it is associated with user terminal IDs that are identification information of the user terminals 20.

The control unit 301 then selects a user terminal 20 of a user to which relocation of the obstruction 401 is requested. The control unit 301 may randomly select from user terminals 20 within a predetermined distance from the obstruction 401, or may select the user terminal 20 closest to the obstruction 401, for example. The control unit 301 may also select a plurality of user terminals 20. Note that an arrangement may be made in which only user terminals 20 of users who are heading in the direction of the obstruction 401 are selected. The direction of movement of the user terminals 20 may be estimated based on the transition of the positions of the user terminal 20, for example, or may be estimated based on the information received regarding results of route navigation being performed at the user terminals 20 or route searches that have been performed at the user terminals 20.

Upon selecting a user terminal 20, the control unit 301 generates a request to relocate the obstruction 401 (hereinafter also referred to as a relocation request). The control unit 301 generates a relocation request such that the user can recognize the obstruction 401. This relocation request includes information regarding the relocation request to be displayed on the display 25 of the user terminal 20, for example. The relocation request may also include an image of the obstruction 401, for example, or position information of the obstruction 401. The control unit 301 then transmits the generated relocation request to the selected user terminal 20.

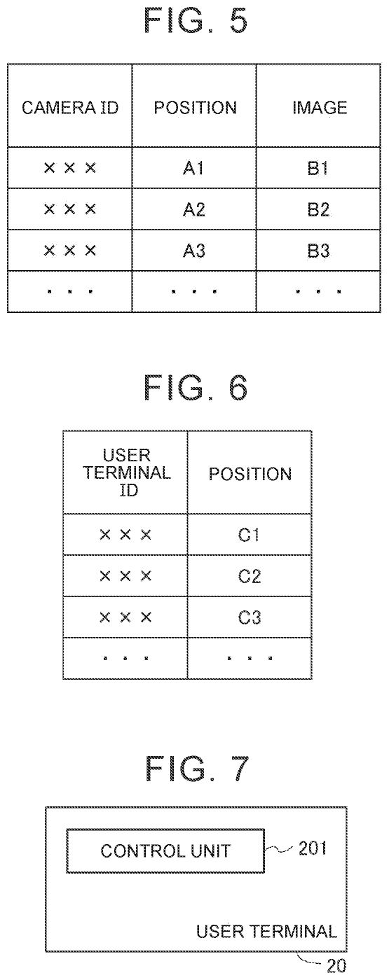

Next, the configuration of the image information stored in the image database 311 will be described with reference to FIG. 5. FIG. 5 is an image exemplifying a table configuration of the image database 311. An image information table has fields of camera ID, position, and image. Identification information unique to the cameras 10 (camera ID) is input to the camera ID field. The camera IDs are imparted to each of the cameras 10 by the control unit 301. Information representing the position of the cameras 10 is input to the position field. The information representing the position of the cameras 10 may be registered in advance, or may be transmitted from the cameras 10 along with the images. Images taken by the cameras 10 are input to the image field. Images are taken by the cameras 10 and transmitted to the server 30 along with camera IDs.

Next, the configuration of position information stored in the user terminal database 312 will be described with reference to FIG. 6. FIG. 6 is a diagram exemplifying a table configuration of the user terminal database 312. A position information table has the fields of user terminal ID and position. Identification information that is unique to the user terminals (user terminal IDs) is input to the user terminal ID field. Information representing the positions of the user terminals 20 is input to the position field. Information representing the positions of the user terminals 20 is transmitted from the user terminals 20 every predetermined amount of time.

Next, the functions of the camera 10 will be described. The camera 10 takes images every predetermined amount of time, for example. Images that are taken are then transmitted to the server 30.

Next, functions of the user terminal 20 will be described. FIG. 7 is a diagram exemplifying a functional configuration of the user terminal 20. The user terminal 20 is provided with a control unit 201 as a functional component. The processor 21 of the user terminal 20 executes processing of the control unit 201 by a computer program in the main storage unit 22. The control unit 201 displays information received from the server 30 on the display 25, for example. That is to say, when receiving a relocation request from the server 30, information regarding the relocation request is displayed on the display 25.

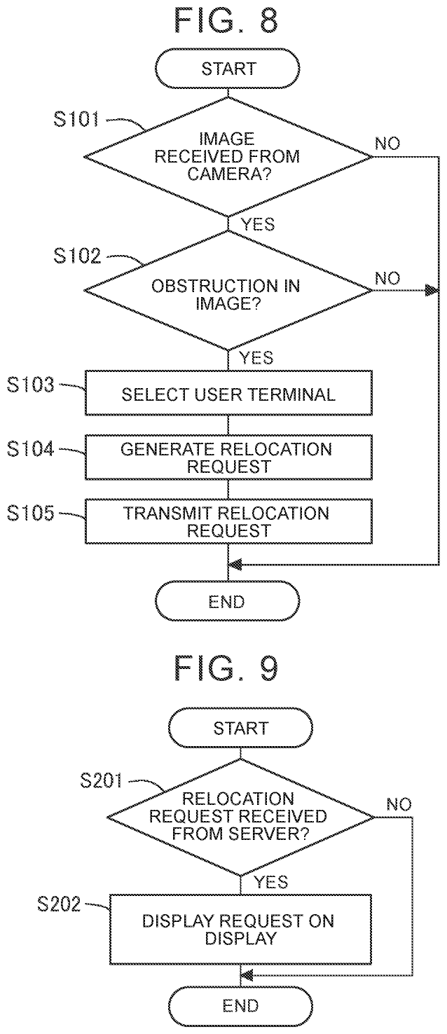

Next, processing of the server 30 transmitting a relocation request to the user terminal 20 will be described. FIG. 8 is a flowchart of processing of the server 30 according to the present embodiment transmitting a relocation request to the user terminal 20. The processing shown in FIG. 8 is executed at the server 30 every predetermined amount of time.

In step S101, the control unit 301 determines whether an image has been received from the camera 10. When a positive determination is made in step S101, the flow advances to step S102, and when a negative determination is made, this routine is ended. In step S102, the control unit 301 determines whether an obstruction 401 is in the image received from the camera 10. The control unit 301 determines whether there is an obstruction 401 in the image by performing image analysis, for example. When a positive determination is made in step S102, the flow advances to step S103, and when a negative determination is made, this routine is ended.

In step S103, the control unit 301 selects the user terminal 20 that the user who is to relocate the obstruction 401 possesses. In this step S103, the user who is to relocate the obstruction 401 is selected by selecting the user terminal 20. The control unit 301 selects the user terminal 20 located at a position that is the closest to the position where the image of the obstruction 401 was taken (or the position of the camera 10), for example. The control unit 301 compares the position information stored in the image database 311 with the position information of the user terminals 20 stored in the user terminal database 312, for example, obtains distances from the obstruction 401 to the user terminals 20, and selects the user terminal 20 of which this distance is the shortest. In this case, the position of the camera 10 and the position of the obstruction 401 may be deemed to be the same. Note that when there is no user terminal 20 within the predetermined distance from the obstruction 401, the present routine may be ended.

In step S104, the control unit 301 generates a relocation request to be transmitted to the user terminal 20. The relocation request may include position information of the camera 10 or the obstruction 401 and image information of the obstruction 401, so as to enable identification of the obstruction 401. Then in step S105, the relocation request is transmitted to the user terminal 20. This user terminal 20 is the user terminal 20 selected in step S103.

Next, processing performed when the user terminal 20 receives the relocation request will be described. FIG. 9 is a flowchart of processing performed when the user terminal 20 according to the present embodiment receives the relocation request from the server 30. The processing shown in FIG. 9 is executed at the user terminal 20 every predetermined amount of time.

In step S201, the control unit 201 determines whether a relocation request has been received from the server 30. When a positive determination is made in step S201, the flow advances to step S202, and when a negative determination is made, this routine is ended. In step S202, the control unit 201 displays information relating to the relocation request on the display 25. For example, the control unit 201 displays the text “Please relocate the obstruction” and an image of the obstruction 401 on the display 25. The control unit 201 may also guide the user over a route to the obstruction 401. For example, the control unit 201 generates a route from the current position of the user terminal 20 to the position of the obstruction 401, which is then displayed on the display 25. The server 30 may generate the route, and transmit the generated route to the user terminal 20.

According to the present embodiment described above, a request to relocate an obstruction 401 is transmitted to a user terminal 20 nearby the obstruction 401, and accordingly the obstruction 401 can be relocated by the user. Accordingly, the visually impaired can be suppressed from coming into contact with the obstruction.

### Second Embodiment

In a second embodiment, users are selected in accordance with the type of obstruction 401, and a relocation request is transmitted to user terminals 20 of these users. For example, when the obstruction 401 is large or when the obstruction 401 is heavy, some users may have difficulty in relocation thereof. In such a case, the control unit 301 selects a user terminal 20 possessed by an adult male, for example, and transmits a relocation request. The control unit 301 determines the type, the size, or the weight of the obstruction 401, for example, by analyzing the images obtained from the camera 10. For example, the control unit 301 may identify the type of the obstruction 401 by related arts, and acquire the weight thereof. The relation of the type and the weight of obstructions 401 is stored in the auxiliary storage unit 33 in advance.

Upon determining the type, the size, or the weight of the obstruction 401, the control unit 301 determines attributes of users capable of relocating this obstruction 401. Attributes of the user include the age or gender of the user, for example. The relation between the type, the size, or the weight of the obstruction 401, and the attributes of users that are able to relocate the obstruction 401, is stored in the auxiliary storage unit 33 in advance. Upon determining the type of the obstruction 401 and the attributes of the user, the control unit 301 stores these in the image database 311.

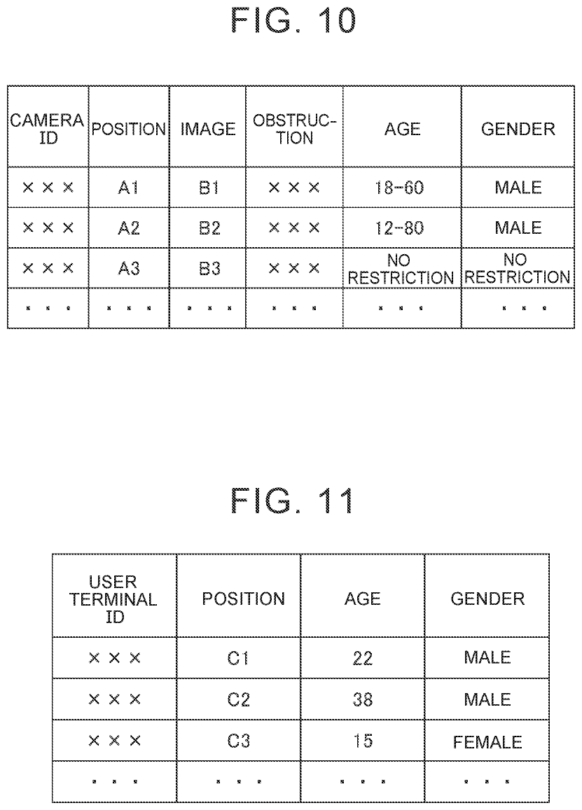

Next, the configuration of image information stored in the image database 311 in the present embodiment will be described with reference to FIG. 10. FIG. 10 is a diagram exemplifying a table configuration of the image database 311 according to the present embodiment. The image information table has the fields of camera ID, position, image, obstruction, age, and gender. The camera ID field, position field, and image field are the same as in FIG. 5, and accordingly description will be omitted. The type, the size, or the weight of the obstruction 401 determined by the control unit 301 is input to the obstruction field. The age of users able to relocate the obstruction 401 is input to the age field. In the example illustrated in FIG. 10, a range of age is input. When there is no restriction regarding age, “no restriction” is input. The relation between the type, the size, or the weight of the obstruction 401, and the age of corresponding users, is stored in the auxiliary storage unit 33. The gender of users able to relocate the obstruction 401 is input to the gender field. In the example illustrated in FIG. 10, when there is no particular restriction regarding gender, “no restriction” is input. The relation between the type, the size, or the weight of the obstruction 401, and the gender of corresponding users, is stored in the auxiliary storage unit 33.

Also, the user may register his/her age in the server 30 in advance, using the user terminal 20. Alternatively, the server 30 may estimate the age of the user using an age distinguishing program based on an image of the user taken by the camera 10.

Next, the configuration of position information stored in the user terminal database 312 according to the present embodiment will be described with reference to FIG. 11. FIG. 11 is a diagram exemplifying a table configuration of the user terminal database 312 according to the present embodiment. A position information table has the fields of user terminal ID, position, age, and gender. An identifier that is unique to the user terminal (user terminal ID) is input to the user terminal ID field. Position information corresponding to the user terminal 20 is input to the position field. Position information corresponding to the user terminal 20 is transmitted from the user terminal 20 every predetermined amount of time. The age of the owner of the user terminal 20 is input to the age field. The gender of the owner of the user terminal 20 is input to the gender field.

Note that information relating to age that is input to the age field, and information relating to gender that is input to the gender field, are input to the user terminal 20 by the user and transmitted from the user terminal 20 to the server 30. Alternatively, an arrangement may be made in which, for example, an image including the user corresponding to the user terminal 20 is identified from position information of the user terminal 20, the position information of the camera 10, and the images transmitted from the camera 10, and the image including the user is analyzed by the control unit 301, thereby estimating the age and the gender of the user corresponding to the user terminal 20. Related arts can be used for this estimation method.

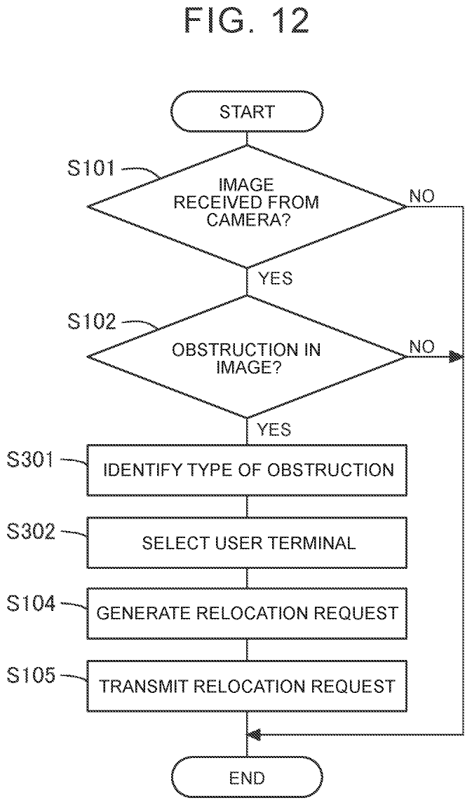

Next, processing of the server 30 transmitting a relocation request to the user terminal 20 will be described. FIG. 12 is a flowchart of processing of the server 30 according to the present embodiment transmitting a relocation request to the user terminal 20. The processing shown in FIG. 12 is executed at the server 30 every predetermined amount of time. Steps executing the same processing as in the flowchart shown in FIG. 8 are denoted by the same signs, and description will be omitted.

When a positive determination is made in step S102 in the flowchart shown in FIG. 12, the flow advances to step S301. In step S301, the control unit 301 identifies the type of the obstruction 401. For example, the control unit 301 identifies the type of the obstruction 401 by analyzing the image including the obstruction 401. The control unit 301 may, for example, extract the obstruction 401 from the image stored in the image database 311, and identify the type of obstruction from features in the image of the obstruction 401 that has been extracted, for example. The relation between features and type of obstruction are stored in the auxiliary storage unit 33 in advance.

In step S302, the control unit 301 selects a user terminal 20 possessed by a user to relocate the obstruction 401. The control unit 301 selects the user terminal 20 possessed by a user who is able to relocate the obstruction 401 and who is at a position closest to the obstruction 401, for example. Whether the user is able to relocate the obstruction 401 is determined in accordance with attributes of the user correlated with the obstruction 401. Accordingly, the control unit 301 acquires the age and the gender of users corresponding to the obstruction 401 from the image database 311. The control unit 301 then acquires the positions, the age of users, and the gender of users, which are correlated with each of the user terminals 20, from the user terminal database 312, and selects the user terminal 20 of a user who is able to handle the obstruction 401. Note that when there is no user terminal 20 of a user who is able to handle the obstruction 401 within a predetermined distance from the obstruction 401, the present routine may be ended. Upon the processing of step S302 being completed, the flow advances to step S104.

According to the present embodiment described above, users to request relocation of the obstruction 401 are selected in accordance with attributes of the obstruction 401 and attributes of the users, and accordingly the probability of having the obstruction 401 relocated is higher.

### Third Embodiment

In a third embodiment, a user who has relocated the obstruction 401 is given a reward. Examples of the reward include e-money, discount coupons, gift certificates, or predetermined points. The discount coupons, gift certificates, or predetermined points may be those usable at shops or the like in the vicinity of the obstruction 401. The user may select a reward from a plurality of candidates.

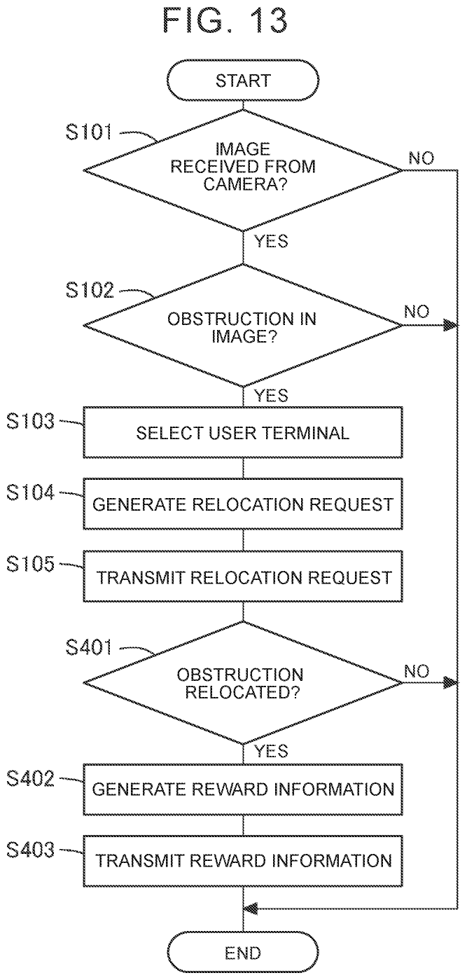

Next, processing of the server 30 giving the user the reward will be described. FIG. 13 is a flowchart of processing of the server 30 according to the present embodiment giving the user a reward. The processing shown in FIG. 13 is executed at the server 30 every predetermined amount of time. Steps executing the same processing as in the flowchart shown in FIG. 8 are denoted by the same signs, and description will be omitted.

In the flowchart in FIG. 13, when the processing of step S105 is complete, the flow advances to step S401. In step S401, the control unit 301 determines whether the obstruction 401 has been relocated. For example, the control unit 301 determines whether the obstruction 401 has been relocated by analyzing images transmitted from the camera 10. At this time, determination may be made regarding whether the user possessing the user terminal 20 to which the relocation request was transmitted has relocated the obstruction 401. When a positive determination is made in step S401, the flow advances to step S402, and when a negative determination is made, this routine is ended. Note that a certain amount of time may be set after ending the processing in step S105, before starting the processing of step S401. This certain amount of time is time necessary for the user to relocate the obstruction 401.

In step S402, the control unit 301 generates reward information. This reward information includes information for the user to receive the reward. The reward information may be stored in the auxiliary storage unit 33 of the server 30 in advance. In step S403, the control unit 301 gives the user the reward by transmitting the reward information to the user terminal 20.

As described above, according to the present embodiment, the user who has relocated the obstruction 401 is given a reward, and accordingly the probability of having the obstruction 401 relocated by the user is higher.

### Fourth Embodiment

In a fourth embodiment, conditions for selecting the user to relocate the obstruction 401 are changed in accordance with situations. Accordingly, the degree of priority of relocation is set for each obstruction 401. For example, when the position of the obstruction 401 is a predetermined position, the degree of priority for the predetermined position is set to be higher than positions other than the predetermined position. The predetermined position here is a position where relocation of the obstruction 401 is highly needed. For example, the predetermined position includes positions where facilities for the visually impaired (e.g., tactile tiles and crosswalks for the visually impaired) are installed. Examples of positions where relocation of the obstruction 401 is highly needed also include locations where traffic of pedestrians or moving bodies (e.g., autonomous vehicles) is heavy. The control unit 301 detects the amount of traffic of pedestrians or moving bodies by analyzing images taken by the camera 10.

Also, the heavier the traffic of pedestrians or moving bodies (e.g., autonomous vehicles) is at a location, the higher the degree of priority may be set, for example. The degree of priority based on the amount of traffic can be acquired by storing the relation between the amount of traffic and the degree of priority in the auxiliary storage unit 33 of the server 30. The control unit 301 may broaden the range of searching for user terminals 20 (predetermined range), or increase the sum of the reward to impart to the user, for example, to further facilitate summoning of users to relocate the obstruction 401 as the degree of priority of the location is higher.

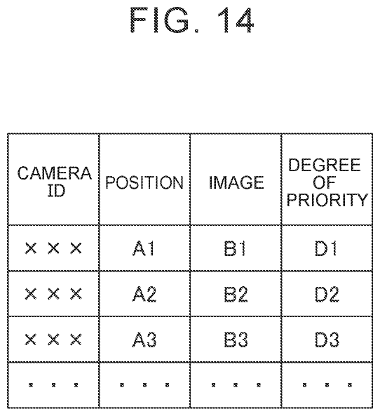

Next, the configuration of image information stored in the image database 311 according to the present embodiment will be described with reference to FIG. 14. FIG. 14 is a diagram exemplifying a table configuration of the image database 311 according to the present embodiment. The image information table has the fields of camera ID, position, image, and degree of priority. The camera ID field, position field, and image field are the same as in FIG. 5, and accordingly description will be omitted. The degree of priority of relocation of the obstruction 401 is input to the degree of priority field. The degree of priority may be expressed in two levels of “high” and “low”, for example, or may be expressed in three or more levels. For example, a place where tactile tiles are installed may be set to a “high” degree of priority, and a place where tactile tiles are not installed may be set to a “low” degree of priority. Places where tactile tiles are installed may be registered in the auxiliary storage unit 33 of the server 30 in advance, or may be determined by analyzing images transmitted from the camera 10.

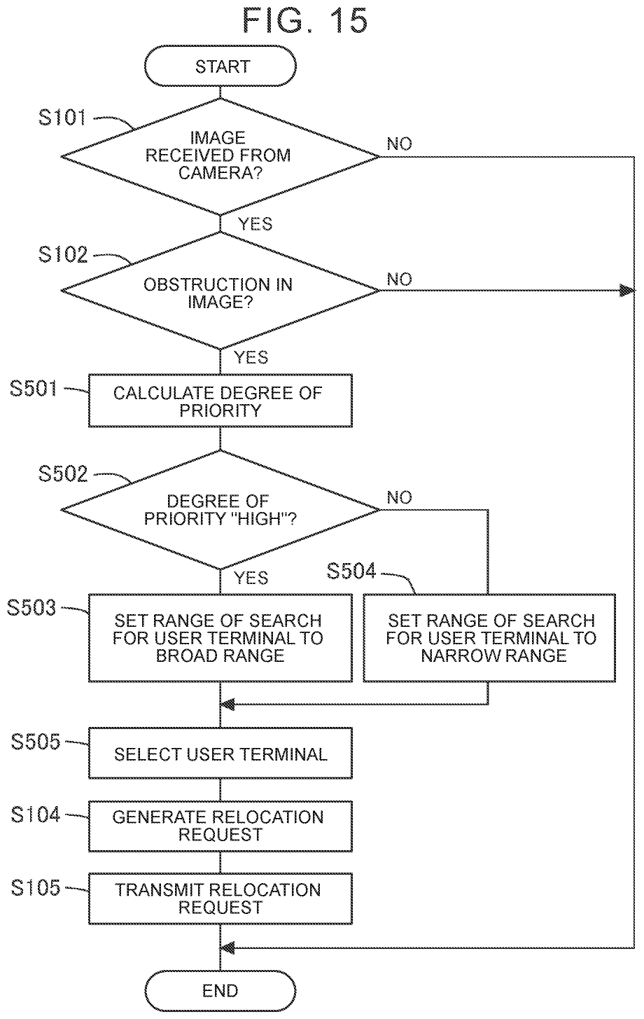

Next, processing of the server 30 transmitting a relocation request to the user terminal 20 will be described. FIG. 15 is a flowchart of processing of the server 30 according to the present embodiment transmitting a relocation request to the user terminal 20. The processing shown in FIG. 15 is executed at the server 30 every predetermined amount of time. Steps executing the same processing as in the flowchart shown in FIG. 8 are denoted by the same signs, and description will be omitted.

When a positive determination is made in step S102 in the flowchart shown in FIG. 15, the flow advances to step S501. In step S501, the control unit 301 calculates the degree of priority of relocating the obstruction 401. The control unit 301 analyzes images transmitted from the camera 10 and acquires the amount of traffic of pedestrians and vehicles, for example. The degree of priority is then calculated based on the amount of traffic. For example, when the amount of traffic is a threshold value or higher, the degree of priority is set to “high”, and when the amount of traffic is below the threshold value, the degree of priority is set to “low”. The threshold value is stored in the auxiliary storage unit 33. Also, for example, the degree of priority is set to “high” for places where tactile tiles are installed, and the degree of priority is set to “low” for places where tactile tiles are not installed.

In step S502, the control unit 301 determines whether the degree of priority is “high”. When a positive determination is made in step S502, the flow advances to step S503, and when a negative determination is made, the flow advances to step S504. In step S503, the control unit 301 sets the search range for user terminals 20 to a broad range. On the other hand, in step S504, the control unit 301 sets the search range for user terminals 20 to a narrow range. Note that the search range set in step S503 is a range that includes all of the search ranges set in step S504 and also is a broader range than the search range set in step S504. In step S505, the control unit 301 selects a user terminal 20 from the search range set in step S503 or in step S504. The selection method of the user terminal 20 is the same as in step S103 in the flowchart shown in FIG. 8.

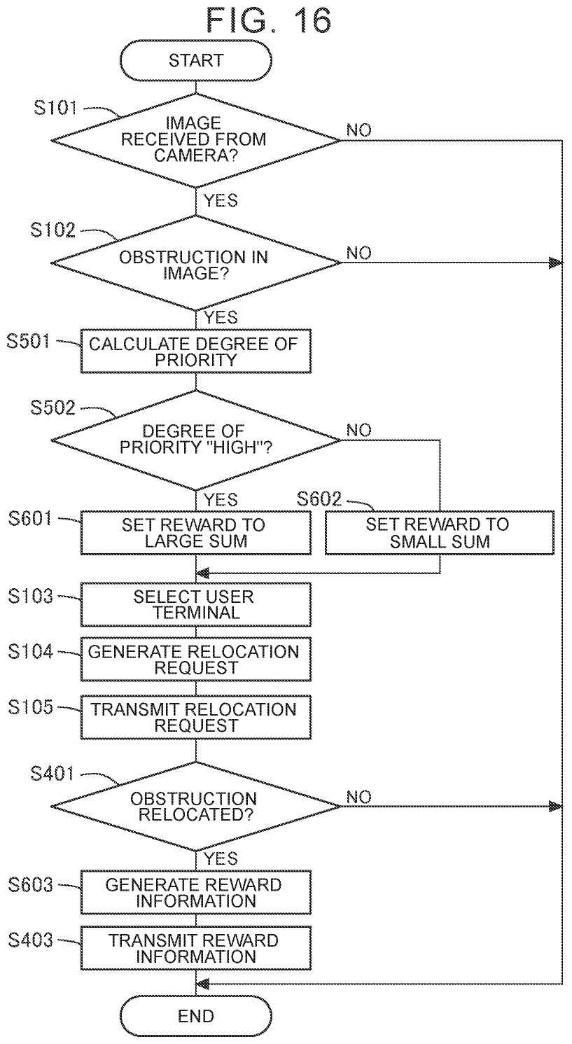

Next, a case of giving a reward to the user who relocates the obstruction 401 will be described. FIG. 16 is a flowchart of processing of the server 30 according to the present embodiment giving a reward to a user. The processing shown in FIG. 16 is executed at the server 30 every predetermined amount of time. Steps executing the same processing as in the flowchart shown in FIG. 8, 13, or 15 are denoted by the same signs, and description will be omitted.

In the flowchart shown in FIG. 16, when a positive determination is made in step S502, the flow advances to step S601, and when a negative determination is made, the flow advances to step S602.

In step S601, the control unit 301 sets the reward to be imparted to a user who relocates the obstruction 401 to a large sum. On the other hand, in step S602, the control unit 301 sets the reward to be imparted to a user who relocates the obstruction 401 to a small sum. The reward set in step S602 is set to a smaller sum than the reward set in step S601.

Also, in the flowchart shown in FIG. 16, when a positive determination is made in step S401, the flow advances to step S603. In step S603, the control unit 301 generates reward information. This reward information is generated in accordance with the sum of the reward set in step S601 or step S602. When the processing of step S603 is complete, the flow advances to step S403.

As described above, according to the present embodiment, the selection range of users is changed, or the reward imparted to the user is varied, in accordance with the degree of priority of relocating the obstruction 401, thereby raising the probability of having the obstruction 401, of which the degree of priority of relocating is high, relocated.

### OTHER EMBODIMENTS

The above embodiments are only exemplary, and the present disclosure can be modified variously without departing from the scope and spirit thereof.

The processes and means described in the present disclosure may be freely combined and carried out, insofar as there is no technical contradiction.

Also, processing described as being performed by one device may be shared and carried out by a plurality of devices. Alternatively, processing described as being performed by different devices may be carried out by a single device. What sort of hardware configuration (server configuration) by which each function is realized in a computer system can be flexibly changed. For example, part of the functions of the server 30 may be provided to the camera 10 or to the user terminal 20.

Although description has been made in the above embodiments that a user receiving the request to relocate the obstruction 401 relocates the obstruction 401, a user receiving the request to relocate the obstruction 401 can decline to relocate the obstruction 401. For example, when the control unit 301 of the server 30 transmits a relocation request to the user terminal 20, the control unit 301 may query the user whether the user can relocate the obstruction 401. When the user does not input a response to the user terminal 20, or responds with a declination to relocate the obstruction 401, the control unit 301 of the server 30 may search for another user to relocate the obstruction 401.

Also, the obstruction 401 is detected based on images taken by the camera 10 in the above embodiments. This camera 10 may be a fixed camera, or may be a camera provided to a moving body. That is to say, an arrangement may be made in which the obstruction 401 is detected, and the position where the obstruction 401 is located is acquired, based on images and position information transmitted from the camera provided to the moving body. Also, instead of detecting the obstruction 401 by analyzing images taken by the camera 10, the obstruction 401 may be detected based on detection values of a pressure sensor installed in the road, for example. That is to say, when there is a predetermined change in pressure, and that state continues for a predetermined amount of time or longer, determination may be made that the obstruction 401 is there. The pressure sensor is capable of detecting the size or weight of the obstruction 401, and accordingly, a user who is appropriate can be selected when selecting a user to relocate the obstruction 401. Further, the obstruction 401 may be detected by radar.

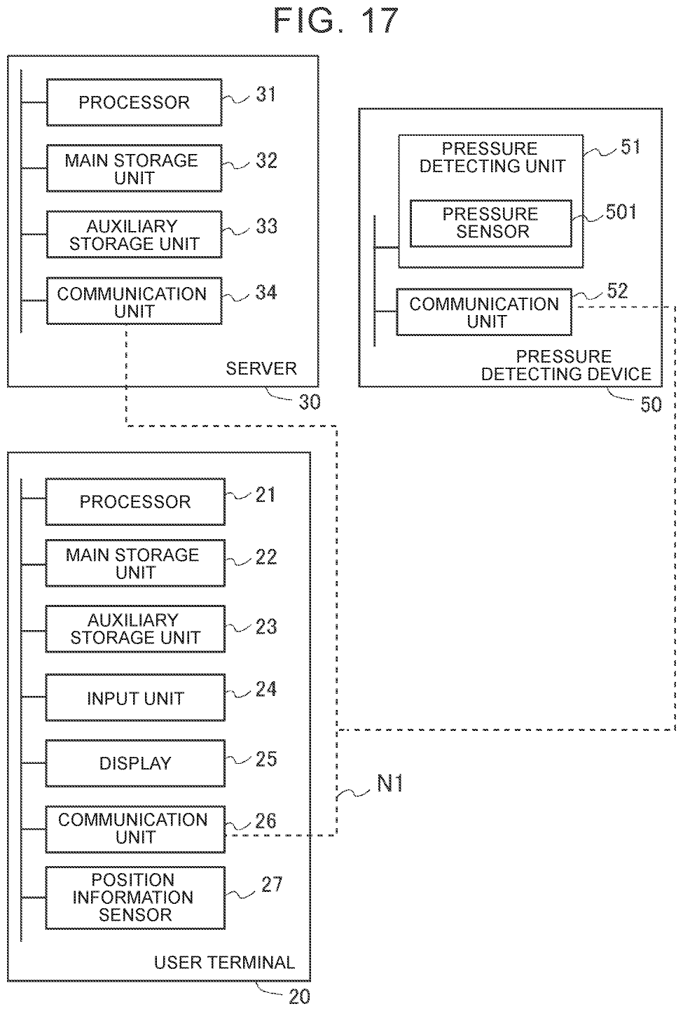

For example, FIG. 17 is a block diagram schematically illustrating an example of configurations of each of a pressure detecting device 50, the user terminal 20, and the server 30, making up the system 1 when detecting whether an obstruction is present based on pressure applied to the road 400. The user terminal 20 and the server 30 are the same as in the above embodiments. The pressure detecting device 50 is provided with a pressure detecting unit 51 and a communication unit 52. The communication unit 52 has the same functions as the communication unit 12 of the camera 10. Also, the pressure detecting unit 51 is configured to include a pressure sensor 501 that detects pressure applied to the road 400. This pressure sensor 501 may be installed at predetermined intervals so as to be able to detect the obstruction 401, for example. In step S101 in FIG. 8, determination is made regarding whether an output value from the pressure sensor 501 is received from the pressure detecting device 50 instead of determining whether an image is received from the camera 10, and in step S102, the control unit 301 determines whether the obstruction 401 is there based on changes in received pressure from the pressure detecting device 50 instead of determining whether the obstruction 401 is in images received from the camera 10.

Also, a relocation request is transmitted to one user with regard to one obstruction 401 in the present embodiment, but the relocation request may be transmitted to a plurality of users instead. Even when a relocation request is transmitted to one user, for example, there is no guarantee that the user will relocate the obstruction 401, and accordingly the relocation request is transmitted to a plurality of users in advance. There also conceivably are cases in which carrying the obstruction 401 by one person alone is difficult, for example. In such cases, the relocation request may be transmitted to as many users as needed to carry the obstruction 401. For example, the larger the obstruction 401 is, or the heaver the obstruction 401 is, the greater the number of users to which the relocation request is transmitted may be. The relation between the size or weight of the obstruction 401 and the number of users may be stored in the auxiliary storage unit 33 in advance.

The present disclosure can also be realized by a computer program implementing the functions described in the above embodiment being supplied to a computer, and one or more processors that the computer has reading and executing the program. Such a computer program may be provided to the computer by a non-transient computer-readable storage medium connectable to a system bus of the computer, or may be provided to the computer via a network. Examples of the non-transient computer-readable storage medium include optional types of disks such as magnetic disks (floppy (registered trademark) disks, HDDs, and so forth), optical discs (compact disc read only memory (CD-ROM) and digital versatile discs (DVD), Blu-ray discs, and so forth), etc., and optional types of media suitable for storing electronic commands such as ROM, RAM, erasable programmable ROM (EPROM), electrically erasable programmable ROM (EEPROM), magnetic cards, flash memory, optical cards, and so forth.

What is claimed is:

1. An information processing device comprising a controller configured to:
detect an obstruction present on a road;
detect a user present within a predetermined distance from the obstruction that is detected; and
transmit, to a terminal of the user that is detected, a request to relocate the obstruction.

2. The information processing device according to claim 1, wherein the controller is configured to:
acquire an image taken by a camera configured to take an image of the road; and
detect the obstruction based on the image.

3. The information processing device according to claim 1, wherein the controller is configured to:
acquire a detection value from a sensor configured to detect pressure applied to the road; and
detect the obstruction based on the detection value from the sensor.

4. The information processing device according to claim 1, wherein the controller is configured to detect the user present within the predetermined distance from the obstruction, by detecting the terminal of the user present within the predetermined distance from the obstruction.

5. The information processing device according to claim 1, wherein the controller is configured to select the terminal of the user that is configured to receive transmission of the request to relocate the obstruction, in accordance with an attribute of the user.

6. The information processing device according to claim 1, wherein the controller is configured to select the terminal of the user that is configured to receive transmission of the request to relocate the obstruction, in accordance with an attribute of the obstruction and an attribute of the user.

7. The information processing device according to claim 1, wherein the controller is configured to transmit information about a reward to the terminal of the user when the obstruction is relocated.

8. The information processing device according to claim 1, wherein the controller is configured to change the predetermined distance in accordance with an amount of traffic of a pedestrian or a vehicle on the road where the obstruction is present.

9. The information processing device according to claim 8, wherein the controller is configured to set the predetermined distance longer as the traffic is heavier.

10. The information processing device according to claim 8, wherein the controller is configured to, when the obstruction is relocated and the controller transmits information about a reward to the terminal of the user, set the reward higher as the traffic is heavier.

11. The information processing device according to claim 1, wherein the controller is configured to change the predetermined distance in accordance with whether a position where the obstruction is present is a predetermined location.

12. The information processing device according to claim 11, wherein the predetermined location is a location at which tactile tiles are installed.

13. The information processing device according to claim 11, wherein:
the controller is configured to set a first distance as the predetermined distance when the position at which the obstruction is present is the predetermined location;
the controller is configured to set a second distance as the predetermined distance when the position at which the obstruction is present is not the predetermined location; and
the first distance is longer than the second distance.

14. The information processing device according to claim 1, wherein:
the controller is configured to set a first value as a reward when the obstruction is relocated, the controller transmits information about the reward to the terminal of the user, and a position at which the obstruction is present is a predetermined location;
the controller is configured to set a second value as the reward when the obstruction is relocated, the controller transmits information about the reward to the terminal of the user, and a position at which the obstruction is present is not the predetermined location; and
the first value is higher than the second value.

15. An information processing method executed by a computer, the information processing method comprising:
detecting an obstruction present on a road;
detecting a user present within a predetermined distance from the obstruction that is detected; and
transmitting, to a terminal of the user that is detected, a request to relocate the obstruction.

16. The information processing method according to claim 15, further comprising selecting, by the computer, the terminal of the user that is configured to receive transmission of the request to relocate the obstruction, in accordance with an attribute of the user.

17. The information processing method according to claim 15, further comprising transmitting, by the computer, information about a reward to the terminal of the user when the obstruction is relocated.

18. An information processing system, comprising:
a sensor configured to output in accordance with an obstruction present on a road; and
a server including a controller, wherein the controller is configured to
detect the obstruction, based on output of the sensor,
detect a user present within a predetermined distance from the obstruction, and
transmit, to a terminal of the user, a request to relocate the obstruction.

19. The information processing system according to claim 18, wherein the controller is configured to select the terminal of the user that is configured to receive transmission of the request to relocate the obstruction, in accordance with an attribute of the user.

20. The information processing system according to claim 18, wherein the controller is configured to transmit information about a reward to the terminal of the user when the obstruction is relocated.
